(19) United States
(12) Patent Application Publication
(10) Pub. No.: US 2013/0217994 A1
(43) Pub. Date: Aug. 22, 2013

(54) METHOD OF GENERATING ELASTICITY DATA, ELASTICITY DATA GENERATING APPARATUS, AND ELASTICITY IMAGE GENERATING SYSTEM BASED THEREON

(71) Applicants: Kyung-hwan Kim, Yongin-si (KR); Na-hyup Kang, Seoul (KR); Tae-hyun Rhee, Yongin-si (KR); Sang-wook Kim, Seoul (KR)

(72) Inventors: Kyung-hwan Kim, Yongin-si (KR); Na-hyup Kang, Seoul (KR); Tae-hyun Rhee, Yongin-si (KR); Sang-wook Kim, Seoul (KR)

(21) Appl. No.: 13/626,197

(22) Filed: Sep. 25, 2012

(30) Foreign Application Priority Data
Feb. 21, 2012 (KR) 10-2012-0017564

(51) Int. Cl. A61B 6/00; A61B 5/103

(52) U.S. Cl. 600/407; 600/587

(57) ABSTRACT

A method of generating elasticity data, an elasticity generating apparatus, and an elasticity image generating system based thereon is provided. The method of generating elasticity data includes receiving first elasticity data of a region of interest (ROI) in a subject, the first elasticity data indicating elasticity corresponding to a predetermined degree of deformation, receiving second elasticity data of a local region of the ROI in the subject, the second elasticity data indicating elasticity corresponding to a local degree of deformation that is greater than the predetermined degree of deformation, and generating third elasticity data of the ROI in the subject based on the received first elasticity data and second elasticity data, the third elasticity data indicating elasticity corresponding to a global degree of deformation that is greater than the predetermined degree of deformation.

### CROSS-REFERENCE TO RELATED APPLICATIONS

This application claims the benefit under 35 U.S.C. §119(a) of Korean Patent Application No. 10-2012-0017564, filed on Feb. 21, 2012, in the Korean Intellectual Property Office, the entire disclosure of which is incorporated herein by reference for all purposes.

### BACKGROUND

1. Field

The following description relates to methods of generating elasticity data, elasticity data generating apparatuses, and elasticity image generating systems based thereon.

2. Description of Related Art

Medical devices configured to create cross-sectional images showing internal structures in a human body, such as ultrasonic imaging devices, X-ray imaging devices, computed tomography (CT) devices, and magnetic resonance imaging (MRI) devices, have been developed to improve convenience and expedite disease diagnosis with respect to a patient.

By way of a probe, ultrasonic imaging devices transmit an ultrasound signal to a predetermined part in a human body from a surface of the human body. The devices obtain an image of blood flow or a section of a soft tissue in the human body based on information determined from an ultrasound echo signal reflected from the soft tissue. Such ultrasonic imaging devices display a reflection coefficient of the ultrasound echo signal as a brightness at each point on a screen to generate a two-dimensional (2D) brightness (B)-mode image. Ultrasonic imaging devices are small, display images in real time, and have no risk of X-ray radiation exposure.

### SUMMARY

In one general aspect, a method of generating elasticity data includes receiving first elasticity data of a region of interest (ROI) in a subject, the first elasticity data indicating elasticity corresponding to a predetermined degree of deformation, receiving second elasticity data of a local region of the ROI in the subject, the second elasticity data indicating elasticity corresponding to a local degree of deformation that is greater than the predetermined degree of deformation, and generating third elasticity data of the ROI in the subject based on the received first elasticity data and second elasticity data, the third elasticity data indicating elasticity corresponding to a global degree of deformation that is greater than the predetermined degree of deformation.

The method may further include that the generating of the third elasticity data includes matching the received first elasticity data and second elasticity data, and interpolating the second elasticity data based on the first elasticity data to generate the third elasticity data.

The method may further include that the second elasticity data indicates elasticity corresponding to a degree of deformation greater than the predetermined degree of deformation in points discretely distributed in the ROI in the subject, and the generating of the third elasticity data further includes interpolating elasticity of adjacent points of the points discretely distributed in the ROI in the subject based on the first elasticity data to generate the third elasticity data.

The method may further include that the generating of the third elasticity data includes calculating an average and a difference between maximum and minimum values of each of the first elasticity data and the second elasticity data, and generating the third elasticity data of the ROI in the subject by modifying the first elasticity data based on the calculated average and difference, and the third elasticity data indicates elasticity corresponding to a degree of deformation greater than the predetermined degree of deformation.

The method may further include that the generating of the third elasticity data is performed by modifying the first elasticity data such that a sum of errors of elasticity values of the second elasticity data and errors of elasticity values of the modified elasticity data is minimized.

The method may further include that the receiving of the second elasticity data includes choosing second elasticity data from elasticity data indicating information about elasticity corresponding to degrees of deformation in a local region of the ROI in the subject, and receiving the chose second elasticity data.

The method may further include that the second elasticity data is obtained by statistical modeling of pieces of elasticity data corresponding to the local degree of deformation that is greater than the predetermined degree of deformation.

The method may further include that the first elasticity data and the second elasticity data indicate elasticity, strain, or a combination thereof.

In another general aspect, there is provided a method of generating an elasticity image of a region of interest (ROI) in a subject, the method including receiving global small deformation elasticity data of the ROI in the subject, the global small deformation elasticity data indicating elasticity corresponding to a predetermined degree of deformation, reading local large deformation elasticity data of a local region corresponding to a portion of the ROI in the subject, the local large deformation elasticity data indicating elasticity corresponding to a local degree of deformation that is greater than the predetermined degree of deformation, generating global large deformation elasticity data of the ROI in the subject from the read local large deformation elasticity data based on the received global small deformation elasticity data, the global large deformation elasticity data indicating elasticity corresponding to a global degree of deformation that is greater than the predetermined degree of deformation, and processing the generated global large deformation elasticity data to generate the elasticity image.

The method may further include that the reading of the local large deformation elasticity data includes detecting a degree of deformation of the ROI in the subject, choosing a piece of the local large deformation elasticity data corresponding to the detected degree of deformation from pieces of the local large deformation elasticity data that indicate elasticity corresponding to degrees of deformation in the local region corresponding to the portion of the ROI in the subject, and reading the chose piece of the local large deformation elasticity data.

In yet another general aspect, there is provided an elasticity data generating apparatus indicating elasticity of a region of interest (ROI) in a subject, the apparatus including a global small deformation elasticity data generating unit configured to generate global small deformation elasticity data of the ROI in the subject, the global small deformation elasticity data indicating elasticity corresponding to a predetermined degree of deformation, a storage unit configured to store local large deformation elasticity data of a local region corresponding to a portion of the ROI in the subject, the local large deformation elasticity data indicating elasticity corresponding to a local degree of deformation that is greater than the predetermined degree of deformation, and a global large deformation elasticity data generating unit configured to generate global large deformation elasticity data of the ROI in the subject from the stored local large deformation elasticity data based on the generated global small deformation elasticity data by receiving the generated global small deformation elasticity data generated by the global small deformation elasticity data generating unit and reading the local large deformation elasticity data stored in the storage unit, the global large deformation elasticity data indicating elasticity corresponding to a global degree of deformation that is greater than the predetermined degree of deformation.

The apparatus may further include that the global large deformation elasticity data generating unit includes a matching unit and an interpolating unit, the matching unit being configured to match the global small deformation elasticity data and the local large deformation elasticity data, the interpolating unit being configured to interpolate the local large deformation elasticity data based on the global small deformation elasticity data to generate the global large deformation elasticity data.

The apparatus may further include that the storage unit is further configured to store the local large deformation elasticity data in points discretely distributed in the ROI in the subject, and the interpolating unit is further configured to interpolate elasticity values of adjacent points of the points discretely distributed in the ROI of the subject into regions between the adjacent points based on the global small deformation elasticity data to generate the global large deformation elasticity data.

The apparatus may further include that the global large deformation elasticity data generating unit generates the global large deformation elasticity data of the ROI in the subject by calculating an average and a difference between maximum and minimum values of each of the elasticity values of the global small deformation elasticity data and the local large deformation elasticity data, and modifying the global small deformation elasticity data based on the calculated average and difference, and the global large deformation elasticity data indicates elasticity corresponding to a degree of deformation greater than the predetermined degree of deformation.

The apparatus may further include that the global large deformation elasticity data generating unit generates the global large deformation elasticity data by modifying the global small deformation elasticity data such that a sum of errors of elasticity values of the local large deformation elasticity data and errors of elasticity values of the modified global small deformation elasticity data is minimized.

The apparatus may further include that the storage unit is further configured to store pieces of the local large deformation elasticity data, wherein the local large deformation elasticity data indicates elasticity corresponding to degrees of deformation, and wherein the global large deformation elasticity data generating unit is further configured to choose one of the pieces of the local large deformation elasticity data, and read the chose one of the pieces.

In still another general aspect, there is provided an elasticity image generating system to generate an elasticity image of a region of interest (ROI) in a subject indicating elasticity, the system including a global small deformation elasticity data generating unit configured to generate global small deformation elasticity data of the ROI in the subject, the global small deformation elasticity data indicating elasticity corresponding to a predetermined degree of deformation, a storage unit configured to store local large deformation elasticity data of a local region corresponding to a portion of the ROI in the subject, the local large deformation elasticity data indicating elasticity corresponding to a local degree of deformation that is greater than the predetermined degree of deformation, and a global large deformation elasticity data generating unit configured to generate global large deformation elasticity data of the ROI in the subject from the stored local large deformation elasticity data based on the generated global small deformation elasticity data by receiving the generated global small deformation elasticity data generated by the global small deformation elasticity data generating unit and reading the local large deformation elasticity data stored in the storage unit, the global large deformation elasticity data indicating elasticity corresponding to a global degree of deformation that is greater than the predetermined degree of deformation, and an image processor configured to process the generated global large deformation elasticity data to generate the elasticity image.

The system may further include a detecting unit configured to detect a degree of deformation of the ROI in the subject, where the global large deformation elasticity data generating unit is further configured to choose one of pieces of the local large deformation elasticity data of the local region corresponding to the portion of the ROI in the subject which corresponds to the detected degree of deformation, and read the chosen piece of local large deformation elasticity data from the storage unit.

In a further general aspect, there is provided a computer-readable recording medium having embodied thereon a program configured to execute a method of generating elasticity data, the method including matching first elasticity data of a region of interest (ROI) in a subject indicating elasticity corresponding to a predetermined degree of deformation and second elasticity data of a local region corresponding to a portion of the ROI in the subject indicating elasticity corresponding to a local degree of deformation that is greater than the predetermined degree of deformation, and generating third elasticity data of the ROI in the subject indicating elasticity corresponding to a global degree of deformation that is greater than the predetermined degree of deformation, the generating comprising interpolating the second elasticity data based on the first elasticity data.

Other features and aspects may be apparent from the following detailed description, the drawings, and the claims.

### BRIEF DESCRIPTION OF THE DRAWINGS

FIG. 1 is a graph illustrating an example of a correlation between stress and strain applied to a liver.

FIG. 2 is a block diagram illustrating an example of an elasticity imaging system.

FIG. 3 is a block diagram illustrating an example of a global large deformation elasticity data generating unit illustrated in FIG. 2.

FIGS. 4A to 4D are graphs illustrating examples of explanations of a method of generating elasticity data.

FIG. 5 is a graph illustrating an example of an enlarged view of portion ‘R’ illustrated in FIG. 4D.

FIG. 6 is a block diagram illustrating another example of an elasticity data generating apparatus.

FIG. 7 is a flowchart illustrating an example of a method of generating an elasticity image.

FIG. 8 is a flowchart illustrating an example of a method of generating elasticity data.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be suggested to those of ordinary skill in the art. In addition, descriptions of well-known functions and constructions may be omitted for increased clarity and conciseness. Expressions such as “at least one of,” when preceding a list of elements, modify the entire list of elements and do not modify the individual elements of the list.

FIG. 2 is a block diagram illustrating an example of an elasticity imaging system 100. Referring to the example illustrated in FIG. 2, the elasticity imaging system 100 includes a probe 210, an elasticity data generating apparatus 200, an image processor 300, an image display device 400, and a user interface 500. Examples of the elasticity imaging system 100 may include an ultrasonic imaging system, a computed tomography (CT) system, and a magnetic resonance imaging (MRI) system.

The elasticity imaging system 100 creates an elasticity image indicating elasticity of a region of interest (ROI) in a body of a patient 110 in order for a medical expert, such as a doctor, to perform a disease diagnosis based on internal structures of the body of the patient 110 displayed by the image. That is, when the medical expert inputs a signal generating an elasticity image via the user interface 500, the probe 210 transmits a source signal to the ROI in the body of the patient 110. Elasticity data that is generated based on a response signal from the body of the patient 110 is processed to generate an elasticity image. Thus, the elasticity imaging system 100 shows the internal structures in the body of the patient 110. In an example of this case, the source signal is a signal known to one of ordinary skill in the art, such as an ultrasound signal and an X-ray signal.

It will be understood by those of ordinary skill in the art that the elasticity image system 100 is not limited to the example illustrated in FIG. 2. In an example, the MRI system creates elasticity data based on signals from internal tissues of a subject lying within a magnetic field generator, in which a high frequency is generated to resonate protons in the body of the subject. Examples of the elasticity image generated by the elasticity imaging system 100 include an ultrasound image, a radiographic image, or an MRI image. Thus, the elasticity image system 100 is not limited to any specific field of medical imaging systems, and may be applied to all medical imaging systems generating elasticity data.

In this regard, the elasticity data indicates information about elasticity of an ROI in the body of the patient 110. Examples of the information about elasticity include strain, elasticity, or information known to one of ordinary skill in the art to be about elasticity. Strain is defined as a ratio of change in length caused when a subject is deformed by stress relative to an original length before the subject is deformed. Elasticity is defined as a ratio of stress to strain. In an example, an elasticity image created by the elasticity imaging system 100 indicates information about elasticity of the ROI in the body of the subject. In another example, the ROI includes internal organs or tissues of the subject. In an example of this case, in an elasticity image illustrating a cross-section of the abdomen of the subject, the ROI includes skin, bones, or a section of the internal organs. In yet another example, the ROI of the elasticity image is a womb of the subject, amniotic fluid, or a section of a fetus.

In an example of generating an elasticity image of internal organs, a medical expert such as a doctor uses an elasticity image indicating elasticity of the internal organs occasionally. In an example of this case, a medical expert uses an elasticity image indicating elastic property of an ROI of a liver in order to diagnose a patient whose liver is in an abnormal condition. In another example, the ROI is a global region or local region of the liver. In an additional example, a local region of the liver includes a linear region or a region contained in a predetermined section of the liver.

In an example, elastic properties of the tissues of the liver are indicated by elasticity. In a general example, elasticity is defined as Young's Modulus (E) represented by Equation 1 below.

$$E = \frac{Stress}{Strain} = \frac{σ}{ε} = \frac{F / {A}_{0}}{Δ L / {L}_{0}} \qquad Equation 1$$

In Equation 1, τ is stress (N/m²), and ε is strain. Stress is defined as force F per unit area A₀ of a subject, and strain is defined as a change in length ΔL caused when the subject is deformed by stress relative to an original length L₀ before the subject is deformed.

With respect to an example of the elasticity imaging system 100 using an ultrasound signal illustrated in FIG. 2, as a result of transmission of an ultrasound signal to the liver of the patient 110 by the probe 210, the liver of the patient 110 contracts in a proceeding direction of the ultrasound signal. Since abnormal tissues, such as a tumor, and normal tissues have different elastic properties, strains thereof are also different. For example, while blood vessels and lobes of the liver have an elasticity that is relatively high, cysts, calcified tissues, and tumors each have a relatively low elasticity. Thus, in an example, a global small deformation elasticity data generating unit 220 generates global small deformation elasticity data GSD that indicates elasticity of the global region of a section of the liver by receiving a reflected ultrasound signal from the probe 210 and detecting strain distribution of internal tissues of the liver.

The ultrasound signal causes small deformations of about several millimeters in the liver. In additional examples, the human liver contracts or relaxes by breathing, heart beating, changes of posture, or other reactions known to one of ordinary skill in the art. Although a degree of deformation varies individually, in an example, the deformation of the human liver generally occurs within several centimeters.

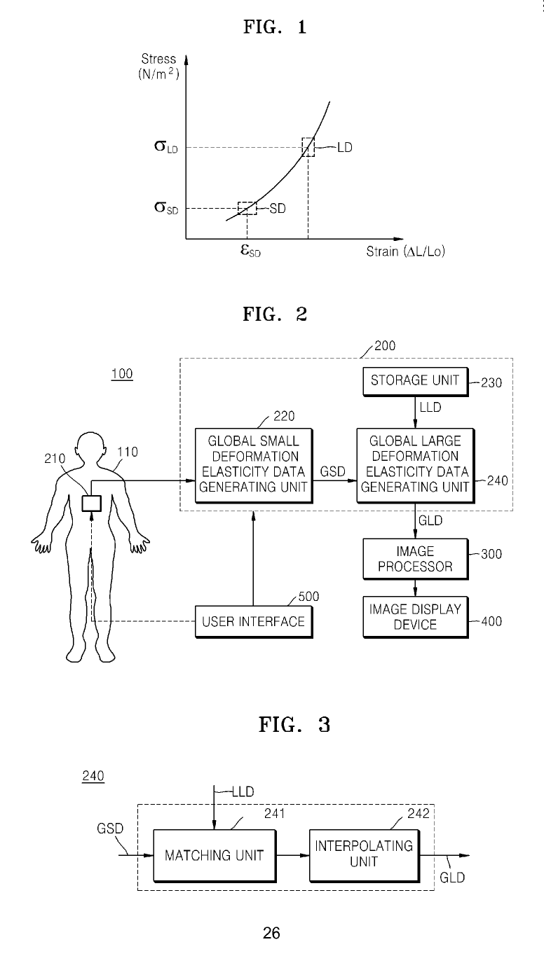

However, elasticity of a point in the liver is not limited. FIG. 1 is a graph illustrating an example of a correlation between stress and strain applied to a liver. Referring to the example illustrated in FIG. 1, strain and stress have nonlinear characteristics. In other words, a ratio (τ_{LD}/ε_{LD}) of stress (τ_{LD}) to strain (ε_{LD}) at a point LD, to which a relatively high strain is applied, is greater than a ratio (τ_{SD}/ε_{SD}) of stress (τ_{SD}) to strain (ε_{SD}) at a point SD, to which a relatively low strain is applied. As such, the ratio of stress to strain in the internal tissues of the liver irregularly changes according to the strain.

Referring further to the example illustrated in FIG. 2, the elasticity data generating apparatus 200 of the elasticity imaging system 100 includes a global small deformation elasticity data generating unit 220, a storage unit 230, and a global large deformation elasticity data generating unit 240. The global small deformation elasticity data generating unit 220 generates global small deformation elasticity data GSD from an electric signal received from the probe 210. That is, the global small deformation elasticity data generating unit 220 generates the global small deformation elasticity data GSD of the global region of the ROI in the subject, where the global small deformation elasticity data GSD indicates elasticity corresponding to a degree of deformation that is relatively small. In an example, the degree of deformation includes strain of the ROI in the subject at a time where elasticity data is to be generated, or elasticity such as Young's modulus. Examples of the global region of the ROI in the subject include a linear region contained in a section of the liver or the entire section of the liver.

The global small deformation elasticity data GSD is input to the global large deformation elasticity data generating unit 240 that generates the global large deformation elasticity data GLD from the global small deformation elasticity data GSD, where the global large deformation elasticity data GLD indicates elasticity corresponding to a degree of deformation that is greater than the global small deformation elasticity data GSD. The storage unit 230 stores local large deformation elasticity data LLD. The local large deformation elasticity data LLD indicates information about elasticity of a local region of the ROI in the subject, where the information corresponds to the degree of deformation that is greater than that of the global small deformation elasticity data GSD. In an example, the local region of the subject is a plurality of points discretely distributed within a one-dimensional or two-dimensional ROI of the subject.

Referring further to the example illustrated in FIG. 2, the storage unit 230 is included in the elasticity data generating apparatus 200, but is not limited thereto. In an example, the storage unit 230 is additionally included in another device outside of the elasticity data generating apparatus 200 as a database. Examples of the storage unit 230 include a hard disc drive, a read only memory (ROM), a random access memory (RAM), a flash memory, or a memory card. In another example, the local large deformation elasticity data LLD is generated by measuring elasticity based on an invasive elasticity measurement when a large deformation occurs in the organs caused by breathing or the heart beating. In an example of this case, the elasticity is measured at a local region corresponding to a portion of the ROI of the organ.

In an example, the local large deformation elasticity data LLD includes at least one sample of elasticity data corresponding to a predetermined degree of deformation of the local region of the subject. In an example of this case, the sample of elasticity data is obtained by measuring strain of a sample model that has a similar size and similar physical characteristics to the organ when a predetermined degree of deformation is applied to the sample model.

In an additional example, a representative image generated by statistical learning based on the at least one sample of elasticity data is determined as the local large deformation elasticity data LLD. Examples of the statistical learning may include an active shape model (ASM), an active appearance model (AAM), and a statistical shape model (SSM). In this regard, the ASM is a statistical model of the shape of objects that iteratively deform to fit to an example of the object in a new image. The AAM is a computer vision algorithm configured to match a statistical model of an object's shape and appearance to a new image. The SSM is geometrical analysis from a set of shapes in which statistics are measured to describe geometrical properties from similar shapes or different groups.

The global large deformation elasticity data generating unit 240 generates global large deformation elasticity data GLD by combining the global small deformation elasticity data GSD received from the global small deformation elasticity data generating unit 220 and the local large deformation elasticity data LLD read from the storage unit 230. That is, the global large deformation elasticity data generating unit 240 generates the global large deformation elasticity data GLD that indicates elasticity corresponding to a large deformation of the global region of the ROI in the subject from the local large deformation elasticity data LLD based on the global small deformation elasticity data GSD.

FIG. 3 is a block diagram illustrating an example of the global large deformation elasticity data generating unit 240 illustrated in FIG. 2. Referring to the example illustrated in FIG. 3, the global large deformation elasticity data generating unit 240 includes a matching unit 241 and an interpolating unit 242. The matching unit 241 matches the global small deformation elasticity data GSD and the local large deformation elasticity data LLD. The matching unit 241 aligns displacements of the global small deformation elasticity data GSD and the local large deformation elasticity data LLD. The interpolating unit 242 interpolates an elasticity value of the local large deformation elasticity data LLD from a local region of the subject into the global region of the ROI of the subject based on the global small deformation elasticity data GSD to generate the global large deformation elasticity data GLD that indicates information about elasticity corresponding to a large deformation in the global region of the ROI of the subject. In an example, the interpolating unit 242 generates global large deformation elasticity data GLD by interpolating elasticity values of adjacent points of the points of the local large deformation elasticity data LLD discretely distributed into regions between the adjacent points based on the elasticity values of the global small deformation elasticity data GSD.

In another example of the global large deformation elasticity data generating unit 240, global large deformation elasticity data GLD is generated that indicates information about elasticity corresponding to a predetermined degree of deformation of the entire ROI of the subject by modifying the global small deformation elasticity data GSD based on analysis results of information about elasticity of the global small deformation elasticity data GSD and the local large deformation elasticity data LLD. In an example of this case, the global large deformation elasticity data generating unit 240 generates global large deformation elasticity data GLD by modifying the global small deformation elasticity data GSD in order to minimize a variation between information about elasticity in the local regions of the local large deformation elasticity data LLD, and information about elasticity obtained by modifying the global small deformation elasticity data GSD.

In a further example, the global large deformation elasticity data generating unit 240 generates the global large deformation elasticity data GLD by modifying the global small deformation elasticity data GSD that indicates information about elasticity in the entire ROI of the subject. The global large deformation elasticity data generating unit 240 extracts elasticity data of a region corresponding to the local region of the local large deformation elasticity data LLD from the global small deformation elasticity data GSD, and compares the extracted elasticity data of the global small deformation elasticity data GSD with deformation information of the local large deformation elasticity data LLD. In this example, the local large deformation elasticity data LLD is deformation information of the local region when a predetermined degree of deformation is applied, and the global small deformation elasticity data GSD is information about elasticity in the entire ROI of the subject when a degree of deformation that is lower than the predetermined degree of deformation is applied.

The global large deformation elasticity data GLD generated by the global large deformation elasticity data generating unit 240 is input to the image processor 300. The image processor 300 processes the global large deformation elasticity data GLD to generate elasticity image data corresponding to the predetermined degree of deformation of the entire ROI of the subject. In an example, the image processor 300 generates an image obtained by combining the global large deformation elasticity data GLD with a brightness (B)-mode ultrasound image. In another example, the elasticity data generating apparatus 200 and the image processor 300 include exclusive chips configured to perform functions of the elements thereof, or exclusive programs stored in a general-purpose central processing unit (CPU) and a storage unit 230.

Image data generated by the image processor 300 is output to the image display device 400. The image display device 400 displays an elasticity image based on the input image data from the image processor 300. Examples of the image display device 400 may include a device configured to display an elasticity image on a screen or paper. The user interface 500 is an interface configured to receive a command or information from a user such as a medical expert. In an example, the user interface 500 is an input device such as a keyboard or a mouse, or a graphic user interface (GUI) displayed in the image display device 400.

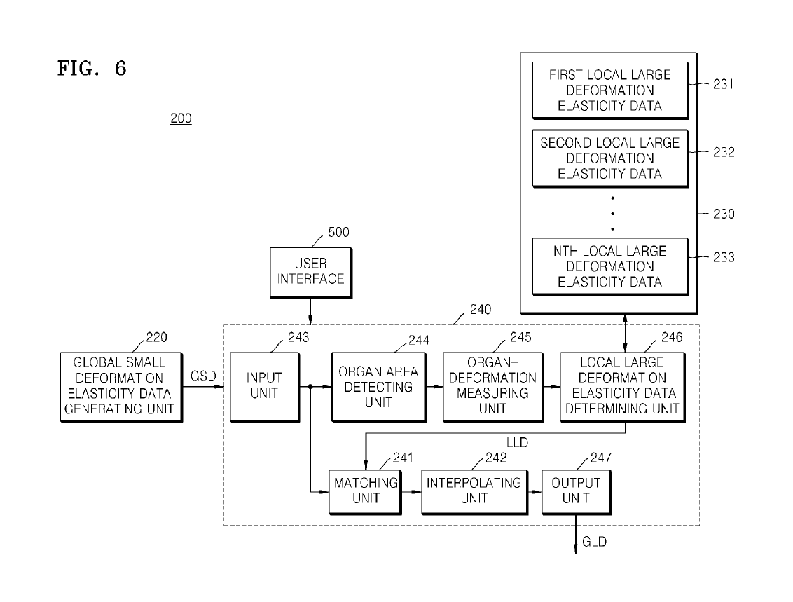

FIG. 6 is a block diagram illustrating an example of an elasticity data generating apparatus 200. The elasticity data generating apparatus 200 is the same in configuration as that of FIG. 2, and thus a description thereof will not be repeated here. The global large deformation elasticity data generating unit 240 included in the elasticity data generating apparatus 200 illustrated in FIG. 6 includes an input unit 243, an organ area detecting unit 244, an organ-deformation measuring unit 245, a local large deformation elasticity data determining unit 246, a matching unit 241, an interpolating unit 242, and an output unit 247.

The input unit 243 is an interface configured to receive the global small deformation elasticity data GSD from the global small deformation elasticity data generating unit 220.

The global small deformation elasticity data GSD input from the input unit 243 is output to the organ area detecting unit 244 and the matching unit 241. The organ area detecting unit 244 detects an organ area by analyzing the global small deformation elasticity data GSD. In an example, the detection of the organ area is performed by determining a boundary between internal and external regions of an organ. In a further example, the organ area detecting unit 244 detects the organ area by detecting a variation in elasticity in an ROI of a subject, and determining a portion having an elasticity variation within a predetermined critical range to be the boundary between the internal and external regions of the organ.

The organ-deformation measuring unit 245 measures the degree of deformation of the organ from the organ area detected by the organ area detecting unit 244. In an example, the organ-deformation measuring unit 245 measures the degree of deformation of the organ by determining a distance between both boundaries of the organ in a linear region as a size of the deformed organ, and comparing the size of the deformed organ with a predetermined size of the organ. When the degree of deformation of the organ is detected, the local large deformation elasticity data determining unit 246 determines local large deformation elasticity data LLD corresponding to the degree of deformation that is the closest to the detected degree of deformation of the organ from among a plurality of pieces of local large deformation elasticity data 231 to 233.

Pieces of local large deformation elasticity data 231 to 233 that indicate information about elasticity corresponding to degrees of deformation of local regions corresponding to portions of the ROI of the subject are stored in the storage unit 230. First to N^{th} pieces of local large deformation elasticity data 231 to 233 indicate information about elasticity corresponding to first to N^{th} degrees of deformation. In an example, the local large deformation elasticity data determining unit 246 determines one of the first to N^{th} degrees of deformation that is the closest to the degree of deformation of the organ detected by the organ-deformation measuring unit 245 and reads the local large deformation elasticity data LLD that indicates information about elasticity corresponding to the determined degree of deformation. The read local large deformation elasticity data LLD is input to the matching unit 241.

The matching unit 241 matches the global small deformation elasticity data GSD and the read local large deformation elasticity data LLD. The interpolating unit 242 generates global large deformation elasticity data GLD by interpolating the local large deformation elasticity data LLD into the entire ROI of the subject based on the global small deformation data GSD. The global large deformation elasticity data GLD generated via interpolation by the interpolating unit 242 is output to the image processor 300 through the output unit 247 that is an interface.

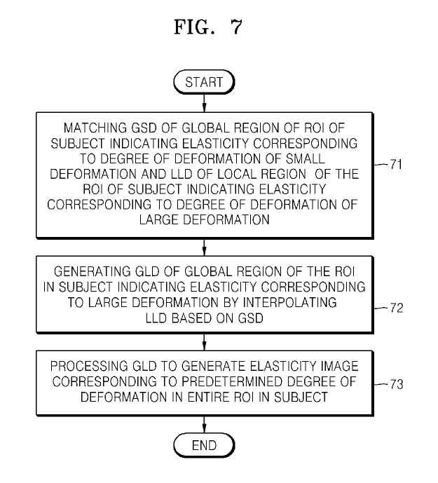

FIG. 7 is a flowchart illustrating an example of a method of generating an elasticity image Referring to the example illustrated in FIG. 7, the global large deformation elasticity data generating unit 240 matches (71) the global small deformation elasticity data GSD of the global region of the ROI of the subject, which indicates elasticity corresponding to the degree of deformation of a small deformation, and the local large deformation elasticity data LLD of the local region of the ROI of the subject, which indicates elasticity corresponding to the degree of deformation of a large deformation.

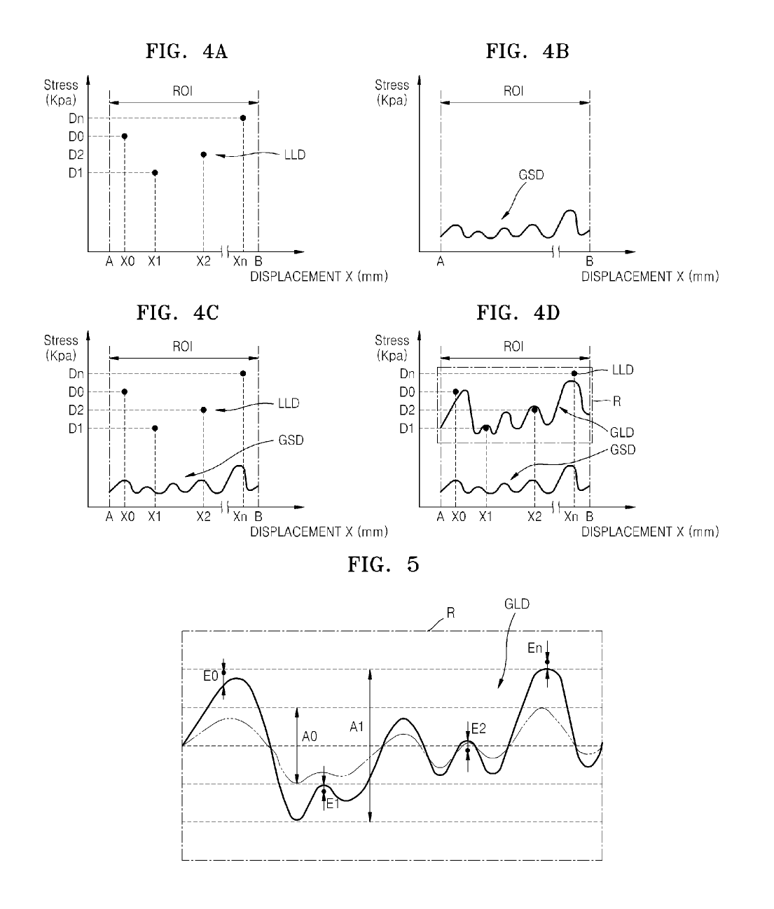

FIGS. 4A to 4D are graphs illustrating examples of explanations of a method of generating elasticity data. Referring to the example illustrated in FIG. 4B, the global small deformation elasticity data GSD shows elasticity with low strain in the ROI corresponding to displacements A to B. Referring to the example illustrated in FIG. 4A, the local large deformation elasticity data LLD shows elasticity with high strain in local regions including displacements X0, X1, X2, . . . , Xn, corresponding to a portion of the ROI of the subject. Stresses D0, D1, D2, . . . , Dn with respect to displacements X0, X1, X2, . . . , Xn of the local large deformation elasticity data LLD illustrated in FIG. 4A are greater than stress of the global small deformation elasticity data GSD illustrated in FIG. 4B since strain applied to obtain the local large deformation elasticity data LLD is greater than strain applied to obtain the global small deformation elasticity data GSD.

The global large deformation elasticity data generating unit 240 generates (72) global large deformation elasticity data GLD of the global region of the ROI in the subject, which indicates elasticity corresponding to a large deformation by interpolating the local large deformation elasticity data LLD based on the global small deformation elasticity data GSD. The generated global large deformation elasticity data GLD is processed (73) to generate an elasticity image corresponding to a predetermined degree of deformation in the entire ROI of the subject.

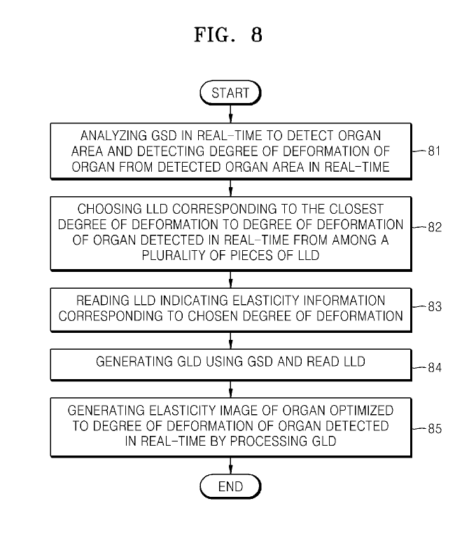

FIG. 8 is a flowchart illustrating an example of a method of generating elasticity data. Referring to the example illustrated in FIG. 8, the global small deformation elasticity data is analyzed (81) to detect an organ area in real-time, and a degree of deformation of the organ is detected from the detected organ area. Local large deformation elasticity data LLD corresponding to a degree of deformation that is the closest to that of the detected degree of deformation is determined (82). Local large deformation elasticity data LLD that indicates information about elasticity corresponding to the determined degree of deformation is read (83) from the storage unit.

The global large deformation elasticity data GLD is generated (84) based on the global small deformation elasticity data GSD and the read local large deformation elasticity data LLD. In an example, elasticity properties in the liver are indicated in consideration of the degree of deformation of the liver that changes in real-time by generating the global large deformation elasticity data GLD by considering the degree of deformation of the organ detected in real-time.

Referring to the example illustrated in FIG. 4C, the global large deformation elasticity data generating unit 240 matches the global small deformation elasticity data GSD and the local large deformation elasticity data LLD. The global large deformation elasticity data generating unit 240 aligns displacements of the global small deformation elasticity data GSD and the local large deformation elasticity data LLD. In an example, the global large deformation elasticity data generating unit 240 generates the global large deformation elasticity data GLD by interpolating the local large deformation elasticity data LLD of the local region of the ROI in the subject into the entire ROI of the subject based on the global small deformation elasticity data GSD. The global large deformation elasticity data generating unit 240 calculates stress of displacement located between two displacements adjacent to the local large deformation elasticity data LLD and supplements stress of the displacement of the local large deformation elasticity data LLD with the calculated stress.

Referring to the example illustrated in FIG. 4D, when displacement X is located between two adjacent points Xₙ₋₁ and Xₙ, stress Dₓ with respect to the displacement X may be interpolated as is shown in Equation 2.

[{Dₙ₋₁×F_{X}×(Xₙ−X)}/{(Xₙ−Xₙ₋₁)Fₙ₋₁}+{Dₙ×F_{X}×(X−Xₙ₋₁)}/{(Xₙ−Xₙ₋₁)Fₙ}] Equation 2

In this regard, Dₙ is stress with respect to Xₙ, Dₙ₋₁ is stress with respect to Xₙ₋₁. In addition, Fₓ is the GSD of displacement X, Fₙ is the GSD of displacement Xₙ, and Fₙ₋₁ is the GSD of displacement Xₙ₋₁.

In an example, the global large deformation elasticity data generating unit 240 generates the global large deformation elasticity data GLD by modifying the global small deformation elasticity data GSD based on analysis results of the global small deformation elasticity data GSD and the local large deformation elasticity data LLD.

FIG. 5 is a graph illustrating an example of an enlarged view of portion ‘R’ illustrated in FIG. 4D. Referring to the example illustrated in FIG. 5, the global large deformation elasticity data generating unit 240 calculates an average and a difference A0 between max and min values of elasticity values of the global small deformation elasticity data GSD, and an average and a difference A1 between max and min values of elasticity values of the local large deformation elasticity data LLD, and shifts the global small deformation elasticity data GSD such that the average of the global small deformation elasticity data GSD is identical to the average of the local large deformation elasticity data LLD. Dashed lines illustrated in FIG. 5 indicate the shifted global small deformation elasticity data.

In an additional example, the global large deformation elasticity data GLD is generated by modifying the elastic values of the global small deformation elasticity data GSD based on a ratio of the difference A1 between the max and min values of elastic values of the local large deformation elasticity data LLD to the difference A0 between the max and min values of the global small deformation elasticity data GSD. The global large deformation elasticity data generating unit 240 determines the global large deformation elasticity data GLD such that an error between the global large deformation elasticity data GLD and the local large deformation elasticity data LLD during the generation of the global large deformation elasticity data GLD is minimized. In an example of this case, the global large deformation elasticity data GLD is generated such that the sum of errors E0, E1, E2, and En with the elasticity values of the local large deformation elasticity data may be minimized.

The units described herein may be implemented using hardware components and software components, such as, for example, microphones, amplifiers, band-pass filters, audio to digital convertors, and processing devices. A processing device may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such as parallel processors. As used herein, a processing device configured to implement a function A includes a processor programmed to run specific software. In addition, a processing device configured to implement a function A, a function B, and a function C may include configurations, such as, for example, a processor configured to implement both functions A, B, and C, a first processor configured to implement function A, and a second processor configured to implement functions B and C, a first processor to implement function A, a second processor configured to implement function B, and a third processor configured to implement function C, a first processor configured to implement function A, and a second processor configured to implement functions B and C, a first processor configured to implement functions A, B, C, and a second processor configured to implement functions A, B, and C, and so on.

The software components may include a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. For example, the software and data may be stored by computer readable recording media. Computer readable recording media may include any data storage device that can store data which can be thereafter read by a computer system or processing device. Examples of computer readable recording media include read-only memory (ROM), random-access memory (RAM), CD-ROMs, magnetic tapes, floppy disks, optical data storage devices. In addition, functional programs, codes, and code segments that accomplish the examples disclosed herein can be easily construed by programmers skilled in the art to which the examples pertain based on and using the flow diagrams and block diagrams of the figures and their corresponding descriptions as provided herein.

Program instructions to perform a method described herein, or one or more operations thereof, may be recorded, stored, or fixed in computer-readable storage media. The program instructions may be implemented by a computer. For example, the computer may cause a processor to execute the program instructions. The media may include, alone or in combination with the program instructions, data files, data structures, and the like. Examples of computer-readable storage media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as that which is produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable storage mediums. In addition, functional programs, codes, and code segments for accomplishing the example embodiments disclosed herein can be easily construed by programmers skilled in the art to which the embodiments pertain based on and using the flow diagrams and block diagrams of the figures and their corresponding descriptions as provided herein. Further, the described unit to perform an operation or a method may be hardware, software, or some combination of hardware and software. For example, the unit may be a software package running on a computer or the computer on which that software is running.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A method of generating elasticity data, comprising:
receiving first elasticity data of a region of interest (ROI) in a subject, the first elasticity data indicating elasticity corresponding to a predetermined degree of deformation;
receiving second elasticity data of a local region of the ROI in the subject, the second elasticity data indicating elasticity corresponding to a local degree of deformation that is greater than the predetermined degree of deformation; and
generating third elasticity data of the ROI in the subject based on the received first elasticity data and second elasticity data, the third elasticity data indicating elasticity corresponding to a global degree of deformation that is greater than the predetermined degree of deformation.

2. The method of claim 1, wherein the generating of the third elasticity data comprises matching the received first elasticity data and second elasticity data, and interpolating the second elasticity data based on the first elasticity data to generate the third elasticity data.

3. The method of claim 2, wherein the second elasticity data indicates elasticity corresponding to a degree of deformation greater than the predetermined degree of deformation in points discretely distributed in the ROI in the subject, and
wherein the generating of the third elasticity data further comprises interpolating elasticity of adjacent points of the points discretely distributed in the ROI in the subject based on the first elasticity data to generate the third elasticity data.

4. The method of claim 1, wherein the generating of the third elasticity data comprises calculating an average and a difference between maximum and minimum values of each of the first elasticity data and the second elasticity data, and generating the third elasticity data of the ROI in the subject by modifying the first elasticity data based on the calculated average and difference, and
wherein the third elasticity data indicates elasticity corresponding to a degree of deformation greater than the predetermined degree of deformation.

5. The method of claim 4, wherein the generating of the third elasticity data is performed by modifying the first elasticity data such that a sum of errors of elasticity values of the second elasticity data and errors of elasticity values of the modified elasticity data is minimized.

6. The method of claim 1, wherein the receiving of the second elasticity data comprises choosing second elasticity data from elasticity data indicating information about elasticity corresponding to degrees of deformation in a local region of the ROI in the subject, and receiving the chose second elasticity data.

7. The method of claim 1, wherein the second elasticity data is obtained by statistical modeling of pieces of elasticity data corresponding to the local degree of deformation that is greater than the predetermined degree of deformation.

8. The method of claim 1, wherein the first elasticity data and the second elasticity data indicate elasticity, strain, or a combination thereof.

9. A method of generating an elasticity image of a region of interest (ROI) in a subject, the method comprising:
receiving global small deformation elasticity data of the ROI in the subject, the global small deformation elasticity data indicating elasticity corresponding to a predetermined degree of deformation;
reading local large deformation elasticity data of a local region corresponding to a portion of the ROI in the subject, the local large deformation elasticity data indicating elasticity corresponding to a local degree of deformation that is greater than the predetermined degree of deformation;
generating global large deformation elasticity data of the ROI in the subject from the read local large deformation elasticity data based on the received global small deformation elasticity data, the global large deformation elasticity data indicating elasticity corresponding to a global degree of deformation that is greater than the predetermined degree of deformation; and
processing the generated global large deformation elasticity data to generate the elasticity image.

10. The method of claim 9, wherein the reading of the local large deformation elasticity data comprises detecting a degree of deformation of the ROI in the subject, choosing a piece of the local large deformation elasticity data corresponding to the detected degree of deformation from pieces of the local large deformation elasticity data that indicate elasticity corresponding to degrees of deformation in the local region corresponding to the portion of the ROI in the subject, and reading the chose piece of the local large deformation elasticity data.

11. An elasticity data generating apparatus indicating elasticity of a region of interest (ROI) in a subject, the apparatus comprising:
a global small deformation elasticity data generating unit configured to generate global small deformation elasticity data of the ROI in the subject, the global small deformation elasticity data indicating elasticity corresponding to a predetermined degree of deformation;
a storage unit configured to store local large deformation elasticity data of a local region corresponding to a portion of the ROI in the subject, the local large deformation elasticity data indicating elasticity corresponding to a local degree of deformation that is greater than the predetermined degree of deformation; and
a global large deformation elasticity data generating unit configured to generate global large deformation elasticity data of the ROI in the subject from the stored local large deformation elasticity data based on the generated global small deformation elasticity data by receiving the generated global small deformation elasticity data generated by the global small deformation elasticity data generating unit and reading the local large deformation elasticity data stored in the storage unit, the global large deformation elasticity data indicating elasticity corresponding to a global degree of deformation that is greater than the predetermined degree of deformation.

12. The apparatus of claim 11, wherein the global large deformation elasticity data generating unit comprises a matching unit and an interpolating unit, the matching unit being configured to match the global small deformation elasticity data and the local large deformation elasticity data, the interpolating unit being configured to interpolate the local large deformation elasticity data based on the global small deformation elasticity data to generate the global large deformation elasticity data.

13. The apparatus of claim 12, wherein the storage unit is further configured to store the local large deformation elasticity data in points discretely distributed in the ROI in the subject, and
the interpolating unit is further configured to interpolate elasticity values of adjacent points of the points discretely distributed in the ROI of the subject into regions between the adjacent points based on the global small deformation elasticity data to generate the global large deformation elasticity data.

14. The apparatus of claim 11, wherein the global large deformation elasticity data generating unit generates the global large deformation elasticity data of the ROI in the subject by calculating an average and a difference between maximum and minimum values of each of the elasticity values of the global small deformation elasticity data and the local large deformation elasticity data, and modifying the global small deformation elasticity data based on the calculated average and difference, and
wherein the global large deformation elasticity data indicates elasticity corresponding to a degree of deformation greater than the predetermined degree of deformation

15. The apparatus of claim 14, wherein the global large deformation elasticity data generating unit generates the global large deformation elasticity data by modifying the global small deformation elasticity data such that a sum of errors of elasticity values of the local large deformation elasticity data and errors of elasticity values of the modified global small deformation elasticity data is minimized.

16. The apparatus of claim 11, wherein the storage unit is further configured to store pieces of the local large deformation elasticity data,
wherein the local large deformation elasticity data indicates elasticity corresponding to degrees of deformation, and
wherein the global large deformation elasticity data generating unit is further configured to choose one of the pieces of the local large deformation elasticity data, and read the chose one of the pieces.

17. An elasticity image generating system to generate an elasticity image of a region of interest (ROI) in a subject indicating elasticity, the system comprising:
a global small deformation elasticity data generating unit configured to generate global small deformation elasticity data of the ROI in the subject, the global small deformation elasticity data indicating elasticity corresponding to a predetermined degree of deformation;
a storage unit configured to store local large deformation elasticity data of a local region corresponding to a portion of the ROI in the subject, the local large deformation elasticity data indicating elasticity corresponding to a local degree of deformation that is greater than the predetermined degree of deformation; and
a global large deformation elasticity data generating unit configured to generate global large deformation elasticity data of the ROI in the subject from the stored local large deformation elasticity data based on the generated global small deformation elasticity data by receiving the generated global small deformation elasticity data generated by the global small deformation elasticity data generating unit and reading the local large deformation elasticity data stored in the storage unit, the global large deformation elasticity data indicating elasticity corresponding to a global degree of deformation that is greater than the predetermined degree of deformation; and
an image processor configured to process the generated global large deformation elasticity data to generate the elasticity image.

18. The system of claim 17, further comprising:
a detecting unit configured to detect a degree of deformation of the ROI in the subject,
wherein the global large deformation elasticity data generating unit is further configured to choose one of pieces of the local large deformation elasticity data of the local region corresponding to the portion of the ROI in the subject which corresponds to the detected degree of deformation, and read the chosen piece of local large deformation elasticity data from the storage unit.

19. A computer-readable recording medium having embodied thereon a program configured to execute a method of generating elasticity data, the method comprising:
matching first elasticity data of a region of interest (ROI) in a subject indicating elasticity corresponding to a predetermined degree of deformation and second elasticity data of a local region corresponding to a portion of the ROI in the subject indicating elasticity corresponding to a local degree of deformation that is greater than the predetermined degree of deformation; and
generating third elasticity data of the ROI in the subject indicating elasticity corresponding to a global degree of deformation that is greater than the predetermined degree of deformation, the generating comprising interpolating the second elasticity data based on the first elasticity data.
